(19) United States
(12) Patent Application Publication
(10) Pub. No.: US 2024/0251491 A1
(43) Pub. Date: Jul. 25, 2024

(54) Gamma Stimulation Apparatus

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 18/626,148

(22) Filed: Apr. 3, 2024

Related U.S. Application Data
(63) Continuation-in-part of application No. 18/613,079, filed on Mar. 21, 2024.
Application No. 18/613,079 is a continuation-in-part of application No. 18/408,523, filed on Jan. 9, 2024.
Application No. 18/408,523 is a continuation-in-part of application No. 18/198,052, filed on May 16, 2023.
Application No. 18/198,052 is a continuation-in-part of application No. 18/101,569, filed on Jan. 25, 2023.
Application No. 18/101,569 is a continuation-in-part of application No. 17/981,123, filed on Nov. 4, 2022.
Application No. 17/981,123 is a continuation-in-part of application No. 17/509,877, filed on Oct. 25, 2021, now abandoned.
Application No. 17/509,877 is a continuation-in-part of application No. 17/148,277, filed on Jan. 13, 2021, now Pat. No. 11,191,863.
Application No. 17/148,277 is a continuation-in-part of application No. 17/094,567, filed on Nov. 10, 2020, now Pat. No. 11,103,612.
Application No. 17/094,567 is a continuation-in-part of application No. 16/180,416, filed on Nov. 5, 2018, now Pat. No. 10,874,762.

(51) Int. Cl. H05B 45/305; A61B 18/00; A61M 21/00; A61N 5/06; H05B 45/37; H05B 45/59

(52) U.S. Cl. CPC H05B 45/305; A61M 21/00; A61N 5/0618; H05B 45/37; H05B 45/59; A61B 2018/00732; A61M 2021/0044; A61N 2005/0626; A61N 2005/0652; A61N 2005/0653

(57) ABSTRACT

A gamma stimulation apparatus comprises a rectifier, a microcontroller, a first modulation operation switch (MOS), a second MOS, and first and second light sources. The microcontroller sends the first MOS a first signal having a first periodical waveform at a first operating frequency (OF1). The first MOS operates the first light source according to the first signal, producing a first light output at the OF1 frequency. The microcontroller sends the second MOS a second signal having a second periodical waveform at a second operating frequency (OF2). The second MOS operates the second light source according to the second signal, producing a second light output at the OF2 frequency. The first and second light outputs superimpose each other to form a superimposed light having a superimposed frequency equal to OF2−OF1 and between 20 Hz and 45 Hz. The superimposed light appears flicker-free (free of flicker) to eyes of a subject.

### BACKGROUND

The present disclosure is a continuation-in-part (CIP) of U.S. patent application Ser. No. 18/613,079, filed 21 Mar. 2024, which is itself is a CIP of U.S. patent application Ser. No. 18/408,523, filed 9 Jan. 2024. Content of aforementioned applications are herein incorporated by reference in their entirety.

### TECHNICAL FIELD

The present disclosure pertains to the field of lighting apparatus and, more specifically, proposes gamma stimulation apparatus.

### DESCRIPTION OF RELATED ART

It has been discovered that by flickering a light at a frequency between 35 Hz to 45 Hz or generating a sound at a similar frequency has the effect of stimulating the cells in certain region of the brain, resulting in using a flicking light or a sound at such a frequency for treating Alzheimer's disease. However, turning on and off a light source at a frequency between 35 Hz to 45 Hz can create visual discomfort to the eyes of a subject. Different approaches have been introduced to overcome this visual discomfort under 40 Hz flickering light.

One of the approaches in U.S. patent application Ser. No. 18/408,523 introduces the use of a controller and two light sources such that the controller would operate these two light sources at two different frequencies resulting a superimposed light operating at a third frequency equal to the difference of these two frequencies. The operating frequency of the first light source is ≥ 50 Hz and the operating frequency of the second light source is greater than the operating frequency of the first light source by at least 30 Hz but no more than 65 Hz. U.S. patent application Ser. No. 18/613,079 introduces a lighting apparatus for generating gamma visual stimulation between 20 Hz and 45 Hz by using a smart controller and only one light source such that the smart controller can operate the one light source according to a superimposed waveform for creating the same gamma stimulation waveform that was created previously by using two light sources each operating at a different frequency. The present disclosure provides further details on the implementation of these lighting apparatuses for gamma stimulation.

### SUMMARY

In one aspect, the gamma stimulation apparatus comprises a rectifier, a microcontroller, a first modulation operation switch (MOS), a second MOS, a first light source, and a second light source. The rectifier is configured to convert an external alternating current (AC) power to an internal direct current (DC) power to power the microcontroller, the first light source, and the second light source. The microcontroller is configured to send the first MOS a first signal having a first periodical waveform at a first operating frequency (OF1), and subsequently, the first MOS (functioning like a switch) is configured to operate the first light source according to the first signal, producing a first light output at the OF1 frequency. Similarly, the microcontroller is configured to send the second MOS a second signal having a second periodical waveform at a second operating frequency (OF2), greater than the OF1 frequency, and subsequently, the second MOS (functioning like a switch) is configured to operate the second light source according to the second signal, producing a second light output at the OF2 frequency. The first light output and the second light output superimpose each other to form a superimposed light having a superimposed frequency equal to OF2−OF1. The superimposed frequency is between 20 Hz and 45 Hz. The superimposed light appears flicker-free (free of flicker) to eyes of a subject.

FIG. 1 shows a first periodical (sinusoidal) waveform at 8 Hz and a second periodical (sinusoidal) waveform as 12 Hz, producing a superimposed waveform (in red) at 4 Hz. FIG. 2 shows a first periodical (trapezoidal) waveform at 8 Hz and a second periodical (trapezoidal) waveform as 12 Hz, producing a superimposed waveform (in red) at 4 Hz. FIG. 3 shows two more periodical trapezoidal waveforms with longer ON state, also producing a superimposed waveform (in red) at 4 Hz. The trapezoidal waveforms in

FIG. 2 and FIG. 3 can be replaced with rectangular waveforms, square waveforms, or triangular waveforms, and they could also produce corresponding superimposed waveforms at 4 Hz.

In some embodiments, the first periodical waveform and the second periodical waveform have a same waveform style, e.g., in sinusoidal waveform, square waveform, rectangular waveform, triangular waveform, or trapezoidal waveform.

In some embodiments, the OF1 frequency is greater than 50 Hz. This is to ensure the baseline frequency is flicker-free to human eyes.

In some embodiments, the superimposed frequency is 40 Hz for this frequency is known to have the best effect of stimulating certain region of the brain for treating Alzheimer's disease. Further in some embodiments, the OF1 frequency is 80 Hz and the OF2 frequency is 120 Hz, yielding a superimposed frequency at 40 Hz.

In some embodiments, the first light source comprises a light emitting diode (LED) or organic LED (OLED), and the second light source comprises an LED or OLED. LED and OLED can be turned on and off quickly and thus are ideal for operating at the OF1 frequency and the OF2 frequency accurately in order to produce a superimposed light with a waveform being a near-perfect superimposition of the first periodical waveform and the second periodical waveform. If the first light source and the second light source have a longer ramp-up or ramp-down time when turning on or off, then the superimposed light, while still has a superimposed frequency equal to OF2−OF1, its waveform would be slightly deviated from the superimposed waveform of the first periodical waveform and the second periodical waveform.

It is foreseeable to use two microcontrollers one for operating the first MOS and the other for operating the second MOS. Thus, in another aspect, the gamma apparatus comprises a rectifier, a first microcontroller, a second microcontroller, a first MOS, a second MOS, a first light source and a second light source. The rectifier is configured to convert an external AC power to an internal DC power to power the first microcontroller, the second microcontroller, the first light source, and the second light source. The first microcontroller is configured to send the first MOS a first signal having a first periodical waveform at a first operating frequency (OF1), and subsequently, the first MOS is configured to operate the first light source according to the first signal, producing a first light output at the OF1 frequency. The second microcontroller is configured to send the second MOS a second signal having a second periodical waveform at a second operating frequency (OF2), greater than the OF1 frequency, and subsequently, the second MOS is configured to operate the second light source according to the second signal, producing a second light output at the OF2 frequency. The first light output and the second light output superimpose each other to form a superimposed light having a superimposed frequency equal to OF2−OF1. The superimposed frequency is between 20 Hz and 45 Hz. The superimposed light appears flicker-free (free of flicker) to eyes of a subject.

In some embodiments, the first periodical waveform and the second periodical waveform have a same waveform style, e.g., in sinusoidal waveform, square waveform, rectangular waveform, triangular waveform, or trapezoidal waveform.

In some embodiments, the OF1 frequency is greater than 50 Hz. This is to ensure the baseline frequency is flicker-free to human eyes.

In some embodiments, the superimposed frequency is 40 Hz for this frequency is known to have the best effect of stimulating certain region of the brain for treating Alzheimer's disease. Further in some embodiments, the OF1 frequency is 80 Hz and the OF2 frequency is 120 Hz, yielding a superimposed frequency at 40 Hz.

In some embodiments, the first light source comprises an LED or OLED, and the second light source comprises an LED or OLED.

In another aspect, the gamma stimulation apparatus comprises a rectifier, a microcontroller, an MOS, and a light source. The rectifier is configured to convert an external AC power to an internal DC power to power the microcontroller and the light source. The microcontroller is configured to send the MOS a signal having a periodical waveform signal at a first frequency (F1) between 20 Hz and 45 Hz, and subsequently, the MOS is configured to operate the light source according to the signal, producing a light output at the F1 frequency. The periodical waveform is decomposable into a first periodical baseline waveform at a second frequency (F2) and a second periodical baseline waveform at a third frequency (F3) such that F1=F3−F2. A light output of the light source appears flicker-free (free of flicker) to eyes of a subject.

In some embodiments, the first periodical baseline waveform and the second periodical baseline waveform have a same waveform style but differ in frequency.

In some embodiments, the periodical waveform has more than one peak within a full cycle.

In some embodiments, the F2 frequency is greater than 50 Hz. This is to ensure the baseline frequency is flicker-free to human eyes.

In some embodiments, the F1 frequency is 40 H for this frequency is known to have the best effect of stimulating certain region of the brain for treating Alzheimer's disease. Further in some embodiments, the F2 frequency is 80 Hz and the F3 frequency is 120 Hz, yielding a superimposed frequency at 40 Hz.

In some embodiments, the light source comprises an LED or OLED.

The microcontroller(s) and the MOS(es) mentioned above may be combined into a control module, whereas the light source(s) stated above may be external, so long as the control module could power the external light source(s) with suitable periodical waveform(s). Thus, in another aspect, a gamma stimulation apparatus comprises a rectifier and a control module having a first power output port and a second power output port. The is configured to convert an external AC power to an internal DC power to power the control module. The control module is configured to output via the first power output port a first output power having a first periodical waveform at a first operating frequency (OF1). The control module is configured to output via the second power output port a second output power having a second periodical waveform at a second operating frequency (OF2). The first power output port is configured to power a first external light source and the second power output port is configured to power a second external light source. A light output of the first external light source and a light output of the second external light source superimpose each other to form a superimposed light having a superimposed frequency equal to OF2−OF1. The superimposed frequency is between 20 Hz and 45 Hz. The superimposed light appears flicker-free (free of flicker) to eyes of a subject.

In some embodiments, the first periodical waveform and the second periodical waveform have a same waveform style, e.g., in sinusoidal waveform, square waveform, rectangular waveform, triangular waveform, or trapezoidal waveform.

In some embodiments, the OF1 frequency is greater than 50 Hz. This is to ensure the baseline frequency is flicker-free to human eyes.

In some embodiments, the superimposed frequency is 40 Hz for this frequency is known to have the best effect of stimulating certain region of the brain for treating Alzheimer's disease. Further in some embodiments, the OF1 frequency is 80 Hz and the OF2 frequency is 120 Hz, yielding a superimposed frequency at 40 Hz.

In yet another aspect, the gamma stimulation apparatus comprises a rectifier and a control module having a power outport. The rectifier is configured to convert an external AC power to an internal DC power to power the control module. The control module is configured to output via the power output port an output power having a periodical waveform signal at a first frequency (F1) between 20 Hz and 45 Hz. The periodical waveform is decomposable into a first periodical baseline waveform at a second frequency (F2) and a second periodical baseline waveform at a third frequency (F3) such that F1=F3−F2. The power output port is configured to power an external light source. A light output of the external light source appears flicker-free (free of flicker) to eyes of a subject.

In some embodiments, the first periodical baseline waveform and the second periodical baseline waveform have a same waveform style but differ in frequency.

In some embodiments, the periodical waveform has more than one peak within a full cycle.

In some embodiments, the F2 frequency is greater than 50 Hz.

In some embodiments, the F1 frequency is 40 Hz. Further in some embodiments, the F2 frequency is 80 Hz. and the F3 frequency is 120 Hz.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts the superimposing of two sinusoidal waveforms with first periodical waveform at 8 Hz and the second periodical waveform at 12 Hz.

FIG. 2 schematically depicts the superimposing of two trapezoidal waveforms with first periodical waveform at 8 Hz and the second periodical waveform at 12 Hz.

FIG. 3 schematically depicts the superimposing of two more trapezoidal waveforms with longer On-state duration.

FIG. 4 schematically depicts an embodiment of the present disclosure using one microcontroller and two light sources.

FIG. 5 schematically depicts an embodiment of the present disclosure using two microcontrollers and two light sources.

FIG. 6 schematically depicts an embodiment of the present disclosure using one microcontroller and one light source.

FIG. 7 schematically depicts an embodiment of the present disclosure using one control module with two power output ports.

FIG. 8 schematically depicts an embodiment of the present disclosure using one control module with one power output port.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of gamma stimulation apparatuses having different form factors.

A gamma stimulation apparatus comprises a rectifier, a microcontroller, a first modulation operation switch (MOS), a second MOS, a first light source, and a second light source. The microcontroller sends the first MOS a first signal having a first periodical waveform at a first operating frequency (OF1), and the first MOS operates the first light source according to the first signal, producing a first light output at the OF1 frequency. The microcontroller sends the second MOS a second signal having a second periodical waveform at a second operating frequency (OF2), and the second MOS operates the second light source according to the second signal, producing a second light output at the OF2 frequency. The first light output and the second light output superimpose each other to form a superimposed light having a superimposed frequency equal to OF2−OF1, and the superimposed frequency is between 20 Hz and 45 Hz. The superimposed light appears flicker-free (free of flicker) to eyes of a subject. Different embodiments of the gamma stimulation apparatuses are also presented.

### Example Implementations

FIG. 4 shows an embodiment of the gamma stimulation apparatus of the present disclosure 100. It comprises a rectifier 101, a microcontroller 102, a first MOS 103, a second MOS 104, a first light source 105, and a second light source 106. The rectifier 101 converts an external AC power (120 Vac at 60 Hz) to an internal DC power (at V2 voltage=24V) to power the microcontroller 102, the first light source 105, and the second light source 106. The microcontroller 102 sends the first MOS 103 a first signal having a first periodical waveform (e.g., a square waveform) at a first operating frequency (OF1) 80 Hz. The first MOS 103 functions like a switch to turn on and off the first light source 105 according to the first signal, producing a first light output at the OF1 frequency 80 Hz. The microcontroller 102 sends the second MOS 104 a second signal having a second periodical waveform (e.g., also a square waveform) at a second operating frequency (OF2) 120 Hz. The second MOS 104 functions like a switch to turn on and off the second light source 106 according to the second signal, producing a second light output at the OF2 frequency 120 Hz. The first light output and the second light output superimpose each other to form a superimposed light having a superimposed frequency equal to OF2−OF1=120 Hz-80 Hz=40 Hz. Such superimposed light appears flicker-free (free of flicker) to eyes of a subject. The first periodical waveform and the second periodical waveform have a save waveform style, i.e., square waveform as shown in FIG. 4, but differ in frequency. The first light source 105 and the second light source 106 comprise an LED or OLED.

In FIG. 4, the first light source 105 and the second light source 106 are shown to operate with square waveforms at V2 voltage=24V. However, it is not required for the first light source 105 and the second light source 106 to operate at the same 24V DC voltage as the microcontroller 102. The present disclosure only requires that the internal DC power to power the microcontroller, the first light source, and the second light source. It is foreseeable to adjust the internal DC power to a different voltage, different from the voltage for powering the microcontroller, to power the first light source and the second light source.

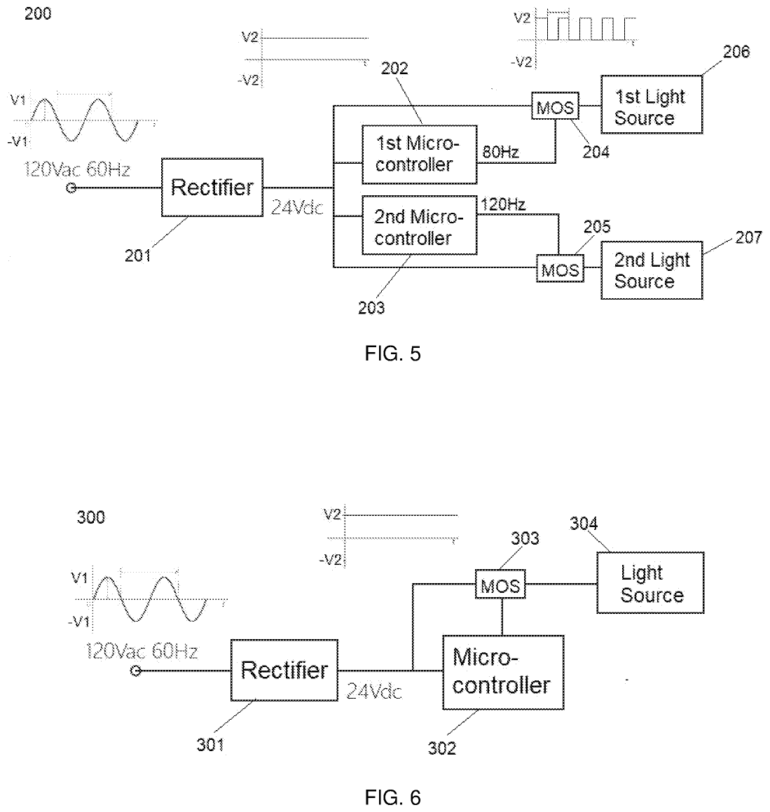

FIG. 5 shows another embodiment of the gamma stimulation apparatus of the present disclosure 200. It comprises a rectifier 201, a first microcontroller 202, a second microcontroller 203, a first MOS 204, a second MOS 205, a first light source 206, and a second light source 207. The rectifier 201 converts an external AC power (120 Vac at 60 Hz) to an internal DC power (at V2 voltage=24V) to power the first microcontroller 202, the second microcontroller 203, the first light source 206, and the second light source 207. The first microcontroller 202 sends the first MOS 204 a first signal having a first periodical waveform (e.g., a square waveform) at a first operating frequency (OF1) 80 Hz. The first MOS 204 functions like a switch to turn on and off the first light source 206 according to the first signal, producing a first light output at the OF1 frequency 80 Hz. The second microcontroller 203 sends the second MOS 205 a second signal having a second periodical waveform (e.g., also a square waveform) at a second operating frequency (OF2) 120 Hz. The second MOS 205 functions like a switch to turn on and off the second light source 207 according to the second signal, producing a second light output at the OF2 frequency 120 Hz. The first light output and the second light output superimpose each other to form a superimposed light having a superimposed frequency equal to OF2−OF1=120 Hz-80 Hz=40 Hz. Such superimposed light appears flicker-free (free of flicker) to eyes of a subject. The first periodical waveform and the second periodical waveform have a save waveform style, i.e., square waveform as shown in FIG. 5 but differ in frequency. The first light source 206 and the second light source 207 comprise an LED or OLED.

In FIG. 5, the first light source 206 and the second light source 207 are shown to operate with square waveforms at V2 voltage=24V. However, it is not required for the first light source 206 and the second light source 207 to operate at the same 24V DC voltage as the first microcontroller 202 and the second microcontroller 203. The present disclosure only requires that the internal DC power to power the first microcontroller, the second microcontroller, the first light source, and the second light source. It is foreseeable to adjust the internal DC power to a different voltage, different from the voltage for powering the first microcontroller and the second microcontroller, to power the first light source and the second light source.

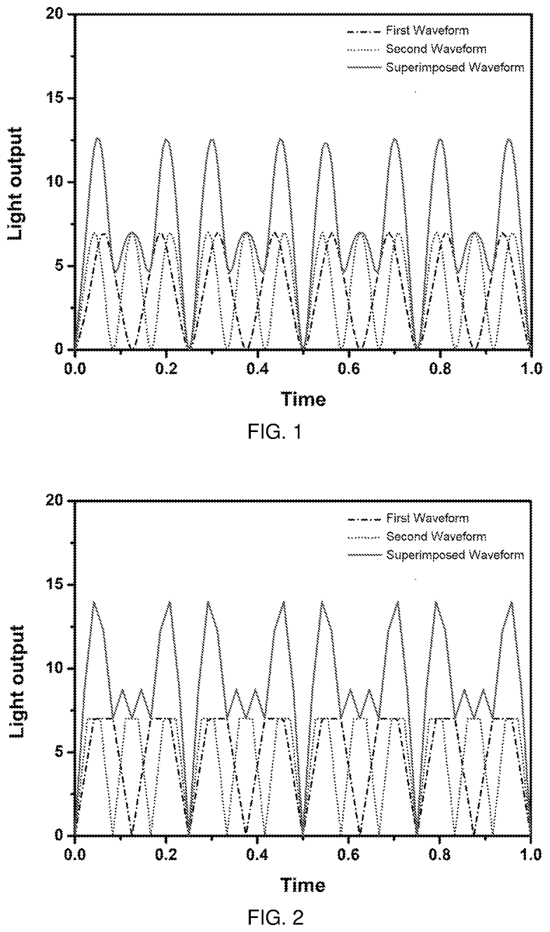

FIG. 6 shows another embodiment of the gamma stimulation apparatus of the present disclosure 300. It comprises a rectifier 301, a microcontroller 302, a MOS 303, and a light source 304. The rectifier 301 is configured to convert an external AC power to an internal DC power (at V2 voltage=24V) to power the microcontroller 302 and the light source 304. The microcontroller 302 sends the MOS 303 a signal having a periodical waveform signal at a first frequency (F1) 40 Hz (like the superimposed waveform in FIG. 1 but at 40 Hz). The MOS 303 functions like a switch to turn on and off the light source 304 according to the signal, producing a light output at the F1 frequency 40 Hz. The periodical waveform is decomposable into a first periodical baseline waveform at a second frequency (F2) 80 Hz (like the first waveform in FIG. 1 but at 80 Hz) and a second periodical baseline waveform at a third frequency (F3) 120 Hz (like the second waveform in FIG. 1 but at 120 Hz) such that F1=F3−F2=120 Hz-80 Hz=40 Hz. A light output of the light source 304 appears flicker-free (free of flicker) to eyes of a subject. As can be seen on the superimposed waveform in FIG. 1, it has two big peaks and one small peak within a full cycle. The light source 304 comprises an LED or OLED.

In FIG. 6, the light source 304 may or may not operate at V2 voltage=24V, the voltage used for powering the microcontroller 302. The present disclosure only requires that the internal DC power to power the microcontroller and the light source. It is foreseeable to adjust the internal DC power to a different voltage, different than the voltage for powering the microcontroller, to power the light source.

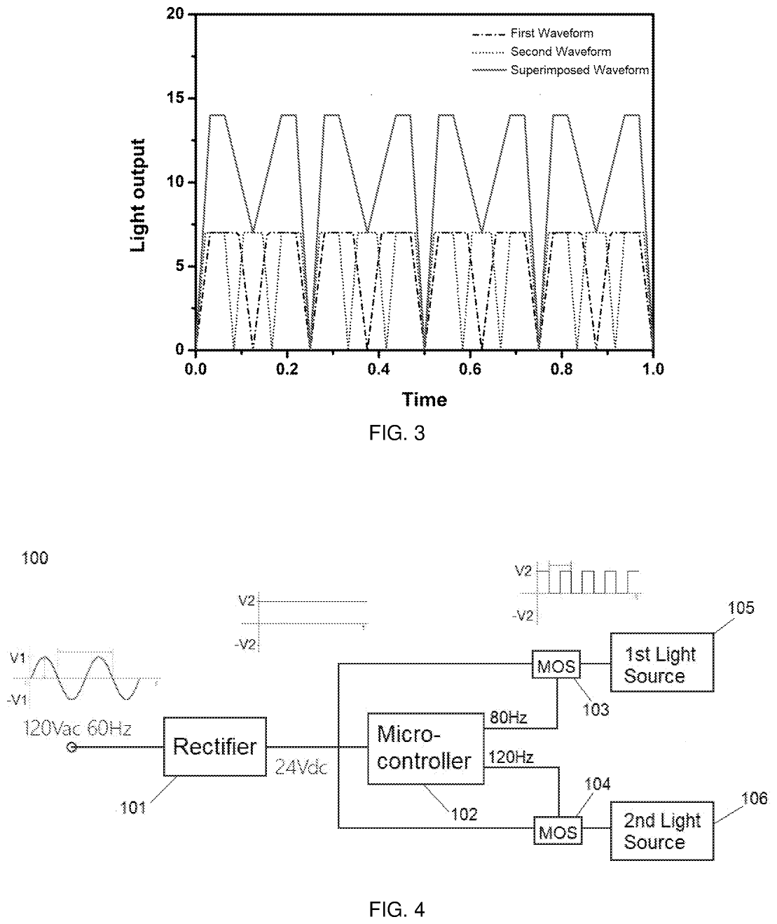

The microcontroller 302 may superimpose internally the first baseline waveform and the second baseline waveform as shown in FIG. 1 to FIG. 3 for creating the superimposed waveform and then instruct the MOS 303 to operate the light source 304 according to the superimposed waveform. Or alternatively, the microcontroller 302 may have the stored data of a periodical waveform as shown by the red waveform in FIG. 1 to FIG. 3 locally (scaled to 40 Hz) and instruct the MOS 303 to operate the light source 304 according to the stored periodical waveform, without doing any superimposition operation of two periodical baseline waveforms.

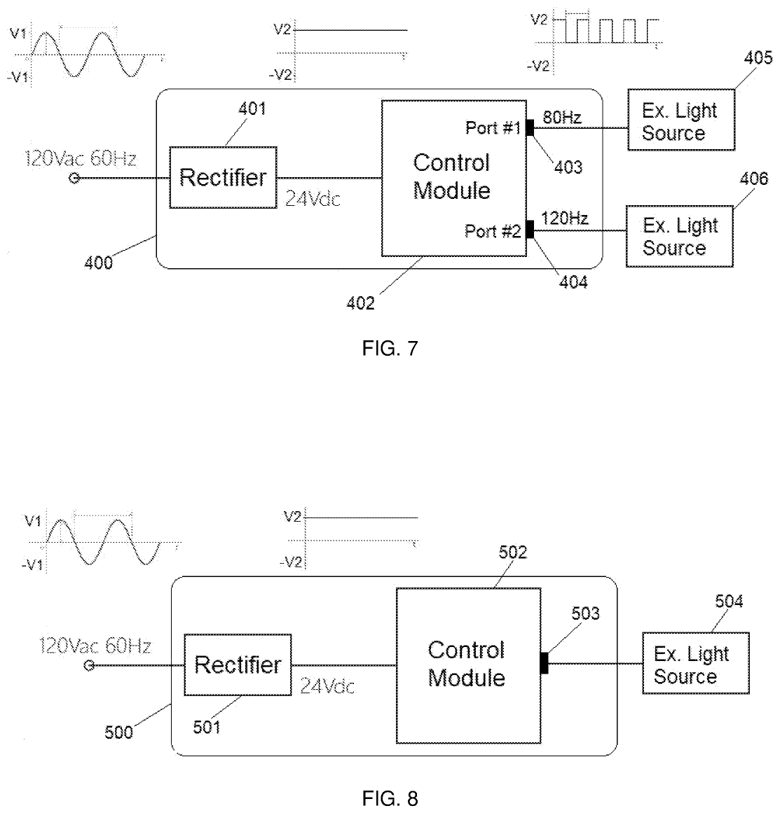

FIG. 7 shows another embodiment of the gamma stimulation apparatus of the present disclosure 400. It comprises a rectifier 401 and a microcontroller 402 having a first power output port 403 and a second power output 404. The rectifier 401 converts an external AC power to an internal DC power (at V2 voltage=24V) to power the control module 402. The control module 402 outputs via the first power output port 403 a first output power having a first periodical waveform (e.g., a square waveform) at a first operating frequency (OF1) 80 Hz. The control module 402 outputs via the second power output port 404 a second output power having a second periodical waveform (e.g., a square waveform) at a second operating frequency (OF2) 120 Hz. The first power output port 403 connects and powers a first external light source 405, and the second power output port 404 connects and powers a second external light source 406. A light output of the first external light source 405 and a light output of the second external light source 406 superimpose each other to form a superimposed light having a superimposed frequency equal to OF2−OF1=120 Hz-80 Hz=40 Hz. Such superimposed light appears flicker-free (free of flicker) to eyes of a subject. The first periodical waveform and the second periodical waveform have a save waveform style, i.e., square waveform as shown in FIG. 7, but differ in frequency.

In FIG. 7, the first external light source 405 and the second external light source 406 are shown to operate with square waveforms at V2 voltage=24V. However, it is not required for the first external light source 405 and the second external light source 406 to operate at the same 24V DC voltage as the control module 402. The present disclosure only requires that the internal DC power to power the control module and that the first power output port to power the first external light source and the second power output power to power the second external light source. It is foreseeable that the voltage powering the control module may be different from the voltage of the first power output port and/or the voltage of the second power output port.

FIG. 8 shows another embodiment of the gamma stimulation apparatus of the present disclosure 500. It comprises a rectifier 501 and a microcontroller 502 having a power output port 503. The rectifier 501 converts an external AC power to an internal DC power (at V2 voltage=24V) to power the control module 502. The control module 502 outputs via its power output port 503 an output power having a first periodical waveform (e.g., a square waveform) at a first frequency F1 40 Hz. The periodical waveform is decomposable into a first periodical baseline waveform at a second frequency (F2) 80 Hz (like the first waveform in FIG. 1 but at 80 Hz) and a second periodical baseline waveform at a third frequency (F3) 120 Hz (like the second waveform in FIG. 1 but at 120 Hz) such that F1=F3−F2=120 Hz-80 Hz=40 Hz. The power output port 503 connects and powers an external light source 504. A light output of the external light source 504 appears flicker-free (free of flicker) to eyes of a subject. As can be seen on the superimposed waveform in FIG. 1, it has two big peaks and one small peak within a full cycle.

In FIG. 8, the external light source 504 may or may not operate at V2 voltage=24V, the voltage used for powering the control module 502. The present disclosure only requires that the internal DC power to power the control module and that the power output port to power an external light source. It is foreseeable that the voltage powering the control module may be different from the voltage of the power output port.

The control module 502 may superimpose internally the first baseline waveform and the second baseline waveform as shown in FIG. 1 to FIG. 3 for creating the superimposed waveform. Or alternatively, the control module 502 may have the stored data of a periodical waveform as shown by the red waveform in FIG. 1 to FIG. 3 locally (scaled to 40 Hz) to operate the external light source 504 according to the stored periodical waveform, without doing any superimposition operation of two periodical baseline waveforms.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A gamma stimulation apparatus, comprising:
a rectifier;
a microcontroller;
a first modulation operation switch (MOS);
a second MOS;
a first light source; and
a second light source,
wherein:
the rectifier is configured to convert an external alternating current (AC) power to an internal direct current (DC) power to power the microcontroller, the first light source, and the second light source,
the microcontroller is configured to send the first MOS a first signal having a first periodical waveform at a first operating frequency (OF1),
the first MOS is configured to operate the first light source according to the first signal to produce a first light output at the OF1 frequency,
the microcontroller is configured to send the second MOS a second signal having a second periodical waveform at a second operating frequency (OF2) greater than the OF1 frequency,
the second MOS is configured to operate the second light source according to the second signal to produce a second light output at the OF2 frequency,
the first light output and the second light output superimpose each other to form a superimposed light having a superimposed frequency equal to OF2−OF1,
the superimposed frequency is between 20 Hz and 45 Hz, and
the superimposed light appears flicker-free to eyes of a subject.

2. The apparatus of claim 1, wherein the first periodical waveform and the second periodical waveform have a same waveform style.

3. The apparatus of claim 1, wherein the OF1 frequency is greater than 50 Hz.

4. The apparatus of claim 1, wherein the superimposed frequency is 40 Hz.

5. The apparatus of claim 4, wherein the OF1 frequency is 80 Hz and the OF2 frequency is 120 Hz.

6. The apparatus of claim 1, wherein the first light source comprises a light emitting diode (LED) or organic LED (OLED), and wherein the second light source comprises another LED or OLED.

7. A gamma stimulation apparatus, comprising:
a rectifier;
a first microcontroller;
a second microcontroller;
a first modulation operation switch (MOS);
a second MOS;
a first light source; and
a second light source,
wherein:
the rectifier is configured to convert an external alternating current (AC) power to an internal direct current (DC) power to power the first microcontroller, the second microcontroller, the first light source, and the second light source,
the first microcontroller is configured to send the first MOS a first signal having a first periodical waveform at a first operating frequency (OF1),
the first MOS is configured to operate the first light source according to the first signal to produce a first light output at the OF1 frequency,
the second microcontroller is configured to send the second MOS a second signal having a second periodical waveform at a second operating frequency (OF2) greater than the OF1 frequency,
the second MOS is configured to operate the second light source according to the second signal to produce a second light output at the OF2 frequency,
the first light output and the second light output superimpose each other to form a superimposed light having a superimposed frequency equal to OF2−OF1,
the superimposed frequency is between 20 Hz and 45 Hz, and
the superimposed light appears flicker-free to eyes of a subject.

8. The apparatus of claim 7, wherein the first periodical waveform and the second periodical waveform have a same waveform style.

9. The apparatus of claim 7, wherein the OF1 frequency is greater than 50 Hz.

10. The apparatus of claim 7, wherein the superimposed frequency is 40 Hz.

11. The apparatus of claim 10, wherein the OF1 frequency is 80 Hz and the OF2 frequency is 120 Hz.

12. The apparatus of claim 7, wherein the first light source comprises a light emitting diode (LED) or organic LED (OLED), and wherein the second light source comprises another LED or OLED.

13. A gamma stimulation apparatus, comprising:
a rectifier;
a microcontroller;
a modulation operation switch (MOS); and
a light source,
wherein:
the rectifier is configured to convert an external alternating current (AC) power to an internal direct current (DC) power to power the microcontroller and the light source,
the microcontroller is configured to send the MOS a signal having a periodical waveform signal at a first frequency (F1) between 20 Hz and 45 Hz,
the MOS is configured to operate the light source according to the signal to produce a light output at the F1 frequency,
the periodical waveform is decomposable into a first periodical baseline waveform at a second frequency (F2) and a second periodical baseline waveform at a third frequency (F3) such that F1=F3−F2, and
a light output of the light source appears flicker-free to eyes of a subject.

14. The apparatus of claim 13, wherein the first periodical baseline waveform and the second periodical baseline waveform have a same waveform style but differ in frequency.

15. The apparatus of claim 13, wherein the periodical waveform has more than one peak within a full cycle.

16. The apparatus of claim 13, wherein the F2 frequency is greater than 50 Hz.

17. The apparatus of claim 13, wherein the F1 frequency is 40 Hz.

18. The apparatus of claim 17, wherein the F2 frequency is 80 Hz. and the F3 frequency is 120 Hz.

19. The apparatus of claim 13, wherein the light source comprises a light emitting diode (LED) or organic LED (OLED).

20. A gamma stimulation apparatus, comprising:
a rectifier; and
a control module having a first power output port and a second power output port,
wherein:
the rectifier is configured to convert an external alternating current (AC) power to an internal direct current (DC) power to power the control module,
the control module is configured to output, via the first power output port, a first output power having a first periodical waveform at a first operating frequency (OF1),
the control module is configured to output, via the second power output port, a second output power having a second periodical waveform at a second operating frequency (OF2),
the first power output port is configured to power a first external light source to produce a first light output,
the second power output port is configured to power a second external light source to produce a second light output superimposing the first light output to form a superimposed light having a superimposed frequency equal to OF2−OF1,
the superimposed frequency is between 20 Hz and 45 Hz, and
the superimposed light appears flicker-free to eyes of a subject.

21. The apparatus of claim 20, wherein the first periodical waveform and the second periodical waveform have a same waveform style.

22. The apparatus of claim 20, wherein the OF1 frequency is greater than 50 Hz.

23. The apparatus of claim 20, wherein the superimposed frequency is 40 Hz.

24. The apparatus of claim 23, wherein the OF1 frequency is 80 Hz and the OF2 frequency is 120 Hz.

25. A gamma stimulation apparatus, comprising:
a rectifier; and
a control module having a power output port,
wherein:
the rectifier is configured to convert an external alternating current (AC) power to an internal direct current (DC) power to power the control module,
the control module is configured to output, via the power output port, an output power having a periodical waveform signal at a first frequency (F1) between 20 Hz and 45 Hz,
the periodical waveform is decomposable into a first periodical baseline waveform at a second frequency (F2) and a second periodical baseline waveform at a third frequency (F3) such that F1=F3−F2, and
the power output port is configured to power an external light source to produce a light output of the external light source appears flicker-free to eyes of a subject.

26. The apparatus of claim 25, wherein the first periodical baseline waveform and the second periodical baseline waveform have a same waveform style but differ in frequency.

27. The apparatus of claim 25, wherein the periodical waveform has more than one peak within a full cycle.

28. The apparatus of claim 25, wherein the F2 frequency is greater than 50 Hz.

29. The apparatus of claim 25, wherein the F1 frequency is 40 Hz.

30. The apparatus of claim 29, wherein the F2 frequency is 80 Hz. and the F3 frequency is 120 Hz.
